(19) United States
(12) Patent Application Publication
(10) Pub. No.: US 2011/0105449 A1
(43) Pub. Date: May 5, 2011

(54) DRY POWDER FORMULATIONS COMPRISING ASCORBIC ACID DERIVATES

(75) Inventor: Jan Trofast, Lund (SE)

(73) Assignee: ASTRAZENECA R&D, Sodertalje (SE)

(21) Appl. No.: 12/740,921

(22) PCT Filed: Nov. 6, 2008
(86) PCT No.: PCT/SE2008/051265

§ 371 Date: Jan. 20, 2011

Related U.S. Application Data
(60) Provisional application No. 60/986,026, filed on Nov. 7, 2007.
(60) Provisional application No. 61/073,443, filed on Jun. 18, 2008.

(51) Int. Cl. A61K 31/58; A61K 31/573; A61K 31/135; A61K 47/22; A61P 11/00; A61P 11/06; A61P 11/08

(52) U.S. Cl. 514/174; 514/180; 514/653; 514/785

(57) ABSTRACT

The invention provides dry powder pharmaceutical formulations comprising an ascorbic acid derivative that demonstrate good inhalation performance and dry powder inhalers containing them.

The present invention relates to dry powder pharmaceutical formulations for use in dry powder inhalers.

Inhalers are well known devices for administering medicinal products to the respiratory tract. They are commonly used for local relief of respiratory diseases such as asthma, bronchitis, chronic obstructive pulmonary disease (COPD), emphysema and rhinitis, but the pulmonary route also provides a conduit for the potential systemic delivery of a variety of medicinal products such as analgesics and hormones. In the treatment of respiratory diseases, because the drug acts directly on the target organ, much smaller quantities of the active ingredient may be used, thereby minimising any potential side effects.

In order to be able to reach the lower respiratory airways, the drug needs to be delivered in finely divided particles or droplets, with an aerodynamic diameter less than 10 micrometers (μm), preferably in the range from 0.5 to 6 micrometers.

There are currently three types of devices used for such delivery: Dry Powder Inhalers (DPIs), pressurised Metered Dose Inhalers (pMDIs) and Nebulisers.

Nebulisers generate a fine aerosol from a solution or suspension of the drug, which is then inhaled. Due to the long administration times, nebulisers are today mainly used for hospital care and also for children who cannot handle inhalers correctly.

Dry Powder Inhalers represent an alternative to pressurised Metered Dose Inhalers that use a volatile propellant to produce an aerosol cloud containing the active ingredient for inhalation.

A finely divided powder for inhalation is light, dusty and fluffy, has poor flowability and is therefore difficult to handle and process, and is notoriously difficult to disperse. For particles with a diameter less than 10 micrometers, electrostatic forces and van der Waals forces are generally greater than the force of gravity, and consequently the material is cohesive. Such powders resist flow under gravity except as large agglomerates. Two main ways of improving powder handling properties whilst maintaining dispersibility can be distinguished: agglomerating the small primary particles into larger loose spheres or adding coarser carrier particles to the small primary particles (to form an ordered mixture). Notwithstanding this, some form of deagglomeration means built into the dry powder inhaler is required to aid dispersion so that an aerosol of respirable particles may be formed. There are many factors that influence powder behaviour, e.g., particle size and distribution, shape, crystallinity, electrostatic charge, chemical composition and environmental humidity. To cope with this, rigorous control of starting materials and processes is required.

Various approaches have been suggested over the years for improving the flowability and dispersibility of dry powder formulations. Elimination of energy-rich “hot spots” on the carrier surface in an ordered mixture leads to lower and more uniform adhesion and cohesion forces, thereby improving dose accuracy and release of fine drug particles. This surface passivation is performed either by using more smooth carrier particles or by adding small particles of a pharmaceutically inactive compound (an additive material) onto the carrier before adding the drug particles onto the modified carrier particles. The most commonly used carrier so far has been lactose, but several experimental attempts have been made to change to other excipients like mannitol, trehalose, amino acids and biodegradable polymers.

The Fine Particle Dose (FPD) of a drug from a dry powder inhaler is a measure of the quantity of drug of effectively deliverable particle size (i.e. with an aerodynamic diameter not greater than 5 to 10 μm) emitted after a single actuation of the DPI. The Fine Particle Fraction (FPF) is the percentage (%) of the emitted dose that the FPD represents. A high FPF is clearly desirable as more of the administered drug will be able to reach the lungs where it can be effective.

The use of an additive material was first mentioned in Published PCT Application No. WO 87/05213 (Chiesi) where the preparation of microgranules of the excipient (lactose) containing a lubricant, such as magnesium stearate or sodium benzoate, was described. This resulted in improved flow and reduced friction of the powder and thereby improved metering of the formulation from a reservoir type dry powder inhaler.

The use of an additive material to improve the fine particle fraction (FPF) was demonstrated by Kassem (London University Thesis, 1990) where the tumbling of lactose carrier particles with 1.5% w/w magnesium stearate or Aerosil 200 (trade mark) colloidal silicon dioxide was shown to enhance the FPF of salbutamol sulfate from a DPI.

Published PCT Application No. WO 96/23485 describes the preparation of dry powder pharmaceutical formulations in which the excipient is mixed with an additive material having anti-adherent or anti-friction properties, consisting of one or more compounds selected from amino acids, phospholipids or surfactants, in order to modulate the adhesive force between the active ingredient and excipient particles. The additive material is stated to be in the form of particles that form a discontinuous covering on the surfaces of the carrier particles. The presence of the additive material is said to promote the release of the small particles of active ingredient from the excipient particles upon actuation of the DPI leading to an increase in the fine particle fraction.

Published PCT Application No. WO 00/53157 describes dry powder pharmaceutical formulations where the carrier particles are coated with lubricant particles at very low concentrations (0.05%-0.5% w) using a mixer. Magnesium stearate is the only lubricant specifically exemplified in the published application although it is suggested that other lubricants such as stearic acid, sodium lauryl sulfate, sodium stearyl fumarate, stearyl alcohol, sucrose monopalmitate and sodium benzoate, may also be suitable depending on the type of carrier and drug used.

Published PCT Application No. WO 01/05429 discloses surface smoothed carrier particles obtained by spraying particles larger than 90 micrometers with water during mixing in an intensive mixer. A lubricant, an anti-adherent agent or a polymer may also be coated onto the carrier, and is applied through dissolution into water/ethanol solution and subsequent spraying onto the carrier particles.

Published PCT Application No. WO 2005/104712 discloses an inhalable dispersible dry powder formulation comprising:
a. a powdered active agent composition comprising an active agent suitable for administration, by inhalation, with a DPI to a subject; and
b. a dissociable powdered carrier comprising sulfoalkyl ether cyclodextrin, wherein the carrier is present in an amount sufficient to aid in release of the active agent from the DPI; wherein
c. the powdered active agent composition has a median particle diameter less than about 37 microns;
d. the carrier has a median particle diameter between about 37 and about 420 microns;
e. active agent and sulfoalkyl ether cyclodextrin are in admixture such that substantially all of the drug is not complexed with the sulfoalkyl ether cyclodextrin; and
f. the active agent composition is dispersed throughout the carrier.

The application describes in general terms a variety of compound classes/compounds and conditions possible to be used with a dissociable powdered carrier comprising sulfoalkyl ether cyclodextrin.

Published PCT Application No. WO 00/028979 describes the use of magnesium stearate in dry powder formulations for inhalation for the purpose of improving the moisture stability and thereby maintaining the FPF when the formulation is tested at higher relative humidity.

Published PCT Application No. WO 02/043702 demonstrates that use of magnesium stearate in dry powder formulations is suitable for delaying the dissolution profile of the drug.

In spite of the treatment of carrier particles with additive materials such as magnesium stearate, magnesium stearate has the disadvantage that it is incompatible with certain types of compounds, for example, compounds containing acid protons or compounds such as aspirin, most vitamins and most alkaloidal salts (Handbook of Pharmaceutical Excipients, 2005). Thus, the need exists for alternative ways of improving the fine particle fraction of dry powder pharmaceutical formulations.

It has now been found that dry powder pharmaceutical formulations containing certain ascorbic acid derivatives demonstrate good inhalation performance as measured by the fine particle fraction.

Furthermore, the choice of ascorbic acid derivative could influence the pharmaceutical profile of the formulation, for example, drug dissolution and chemical stability. In treating respiratory disorders it could be an advantage to have a fast onset of action of the drug, for example, in order to prevent or treat an acute asthma attack.

Being that the antioxidant property of ascorbic acid is well known since it is used as a preservative in pharmaceutical products and foodstuffs, the formulations according to the invention have the advantage of possessing a high degree of stability to chemical degradation.

In accordance with the present invention, there is therefore provided a dry powder formulation for use in inhalation therapy comprising a pharmaceutically active substance, an excipient and an additive being the reaction product of ascorbic acid with (i) a saturated or unsaturated, straight or branched C₁₂-C₁₈ fatty acid, (ii) a straight or branched C₈-C₁₈ alkyl or alkenyl mono ester of a dibasic acid, (iii) a straight or branched C₁₀-C₁₈ alkanoyl or alkenoyl N-substituted amino acid, or (iv) a straight or branched C₁₀-C₁₈ alkanoyl or alkenoyl ester of a hydroxy acid.

The invention further provides a dry powder formulation for use in inhalation therapy comprising a pharmaceutically active substance, an excipient and an additive being the reaction product of ascorbic acid with (i) a saturated or unsaturated, straight or branched C₁₂-C₁₈ fatty acid, (ii) a straight or branched C₈-C₁₈ alkyl or alkenyl mono ester of a dibasic acid, (iii) a straight or branched C₁₀-C₁₈ alkanoyl or alkenoyl N-substituted amino acid, or (iv) a straight or branched C₁₀-C₁₈ alkanoyl or alkenoyl ester of a hydroxy acid, provided that the excipient is not a cyclodextrin or any derivative (including a sulfoalkyl ether derivative) thereof.

The present invention also provides the use of an additive being the reaction product of ascorbic acid with (i) a saturated or unsaturated, straight or branched C₁₂-C₁₈ fatty acid, (ii) a straight or branched C₈-C₁₈ alkyl or alkenyl mono ester of a dibasic acid, (iii) a straight or branched C₁₀-C₁₈ alkanoyl or alkenoyl N-substituted amino acid, or (iv) a straight or branched C₁₀-C₁₈ alkanoyl or alkenoyl ester of a hydroxy acid, in a dry powder formulation for use in inhalation therapy in order to increase fine particle dose.

The present invention still further provides a carrier material suitable for use in a dry powder pharmaceutical formulation comprising an excipient mixed with an additive being the reaction product of ascorbic acid with (i) a saturated or unsaturated, straight or branched C₁₂-C₁₈ fatty acid, (ii) a straight or branched C₈-C₁₈ alkyl or alkenyl mono ester of a dibasic acid, (iii) a straight or branched C₁₀-C₁₈ alkanoyl or alkenoyl N-substituted amino acid, or (iv) a straight or branched C₁₀-C₁₈ alkanoyl or alkenoyl ester of a hydroxy acid.

The additive used in the formulations of the invention may be the reaction product of ascorbic acid with a saturated or unsaturated, straight or branched C₁₂-C₁₈, or C₁₄-C₁₈, or C₁₆-C₁₈, fatty acid, examples of which include ascorbyl dodecanoate (laurate), ascorbyl myristate, ascorbyl palmitate and ascorbyl stearate.

In one embodiment of the invention, the additive is ascorbyl palmitate, especially 6-O-palmitoyl-L-ascorbic acid.

In another embodiment, the additive is the reaction product of ascorbic acid with a straight or branched C₈-C₁₈ alkyl or alkenyl mono ester of a dibasic acid such as fumaric acid, maleic acid, succinic acid, malonic acid or malic acid. Examples of such monoesters include

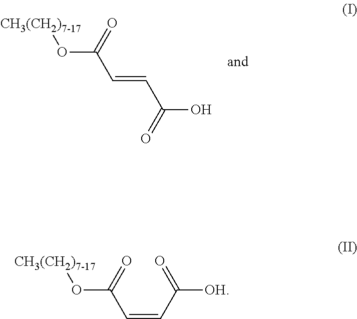

In still another embodiment, the additive is the reaction product of ascorbic acid with a straight or branched C₁₀-C₁₈ alkanoyl or alkenoyl N-substituted amino acid such as leucine. Examples of such substituted amino acids include

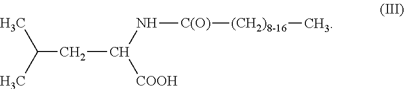

In yet another embodiment, the additive is the reaction product of ascorbic acid with a straight or branched C₁₀-C₁₈ alkanoyl or alkenoyl ester of a hydroxy acid such as lactic acid. Examples of such esters include

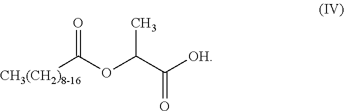

The additive may be present in an amount from 0.5 to 15 or 20, e.g. from 0.5 or 1 or 1.5 or 2 or 2.5 or 3 or 3.5 or 4 or 4.5 to 5 or 6 or 7 or 8 or 9 or 10 or 11 or 12 or 13 or 14 or 15 or 20, percent by weight (% w) based on the total weight of the formulation.

In an embodiment of the invention, the additive is present in an amount from 0.5 to less than 2% w, e.g. from 0.5 to 1 or 1.5% w.

In another embodiment, the additive is present in an amount from greater than 2 to 10% w, e.g. from 2.5 to 3 or 3.5 or 4 or 4.5 or 5 or 6 or 7 or 8 or 9 or 10% w.

In a further embodiment, the additive is present in an amount from 5 to 10% w, in particular 10% w.

Without being bound to any particular theory, the additive is believed to reduce the adhesive force between the particles of pharmaceutically active substance and excipient, so facilitating deaggregation and dispersion of the active substance during aerosolisation.

The excipient will comprise any pharmacologically inert material or combination of materials that is acceptable for inhalation. Examples of excipients that may be used include saccharides such as glucose, galactose, D-mannose, arabinose, sorbose, lactose, maltose, sucrose or trehalose, and sugar alcohols such as mannitol, maltitol, xylitol, sorbitol, myo-inositol and erythritol. Solvates (e.g. hydrates) of these compounds may be used where such exist.

In an embodiment of the invention, the excipient is lactose or lactose monohydrate (in particular α-lactose monohydrate) or a mixture thereof.

In another embodiment of the invention, the excipient is erythritol.

The excipient will be present in the formulation of the invention in an amount of at least 70 percent by weight (% w), e.g. in the range from 70 or 80 to 90 or 95 or 99% w, based on the total weight of the formulation.

In an embodiment of the invention, the excipient is used in an amount of 80 or 81 or 82 or 83 or 84 or 85 to 86 or 87 or 88 or 89 or 90 or 91 or 92 or 93 or 94 or 95 or 96 or 97 or 98 or 99% w.

The excipient particles will generally have a mass median diameter (MMD) equal to or greater than 20 micrometers (μm), e.g. a mass median diameter in the range from 20 to 150 micrometers (μm).

There are several particle sizing methods available that can be used to obtain, directly or after recalculation, geometrical particle size distributions, see for example “Powder sampling and particle size measurement” by T. Allen, Elsevier, Netherlands, 2003. Laser light scattering is just one example of such methods.

The mass median diameter is defined as the particle diameter for which 50 percent by weight of the particles are smaller than this diameter and 50 percent by weight are larger.

As for the delivery of the fine drug particles to the lungs, the aerodynamic diameter and the fine particle dose are the more relevant measures, and can be measured using an impinger, as described in United States Pharmacopoeia 30, section <601> or in Eur. Pharmacopoeia 5.8 section 2.9.18.

As concerns the particle sizes of the constituents of the formulations, however, geometric particle size distributions are relevant and are most commonly used.

If desired, the formulations of the present invention may contain two or more excipient particle size ranges. For example, the excipient may consist of two components having different particle size distributions, a fine component and a coarse component. The fine component may be of the same material as the coarse component but may, alternatively, be of a different material. The fine component may be used in an amount in the range from 2 to 20 percent by weight (% w) based on the total weight of the formulation and may have a MMD equal to or less than 20 micrometers (μm), e.g. in the range from 0.5 to 20 μm, particularly from 2 to 10 μm, whilst the coarse component may have a MMD in the range from 30 or 50 to 70, 90 or 100 micrometers (μm), for example, from 30 to 70 μm.

As described in the review article entitled “The Influence of Fine Excipient Particles on the Performance of Carrier-Based Dry Powder Inhalation Formulations” in Pharmaceutical Research, 2006, 23(8), pages 1665 to 1674, the inclusion of a small amount of fine particle excipient in a carrier-based dry powder inhalation system is a well researched technique to improve formulation performance by increasing the fine particle dose.

The pharmaceutically active substance can be any therapeutic molecule in dry powder form that is suitable for administration by the inhalation route. For administration by the inhalation route, the particles of active substance will generally have a MMD of equal to or less than 5 micrometers (μm), e.g. in the range from 0.1 or 0.5 or 1 to 5 μm, and in particular a MMD equal to or less than 3 micrometers (μm), e.g. in the range from 0.1 or 0.5 or 1 to 3 μm. Particles of active substance of the desired size are prepared by micronisation, for example, using techniques known in the art such as milling, or controlled precipitation, supercritical fluid and spray drying methodologies. Such known techniques are described, for example, in the article by Rasenack et al. entitled “Micron-size Drug Particles: Common and Novel Micronization Techniques” in Pharmaceutical Development and Technology, (2004), 9(1), pages 1 to 13.

Examples of pharmaceutically active substances that may be used include

(a) glucocorticosteroids such as budesonide, fluticasone (e.g. as propionate ester or furoate ester), mometasone (e.g. as furoate ester), beclomethasone (e.g. as 17-propionate or 17,21-dipropionate esters), ciclesonide, triamcinolone (e.g. as acetonide), flunisolide, zoticasone, flumoxonide, rofleponide, ST 126, loteprednol (e.g. as etabonate), etiprednol (e.g. as dichloroacetate), butixocort (e.g. as propionate ester), prednisolone, prednisone, tipredane, steroid esters according to WO 2002/12265, WO 2002/12266 and WO 2002/88167, e.g., 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl)ester and 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, and steroid esters according to DE 4129535;
(b) long-acting β₂-agonists such as salmeterol, formoterol, bambuterol, carmoterol, indacaterol, GSK 159797, formanilide derivatives e.g. 3-(4-{[6-({(2R)-2-[3-(formylamino)-4-hydroxyphenyl]-2-hydroxyethyl}amino)hexyl]oxy}-butyl)-benzenesulfonamide as disclosed in WO 2002/76933, benzenesulfonamide derivatives e.g. 3-(4-{[6-({(2R)-2-hydroxy-2-[4-hydroxy-3-(hydroxy-methyl)phenyl]ethyl}amino)-hexyl]oxy}butyl)benzenesulfonamide as disclosed in WO 2002/88167, aryl aniline compounds as disclosed in WO 2003/042164 and WO 2005/025555 and indole derivatives as disclosed in WO 2004/032921; and
(c) anticholinergic compounds such as ipratropium (e.g. as bromide), tiotropium (e.g. as bromide), oxitropium (e.g. as bromide), tolterodine, aclidinium (e.g. as bromide), glycopyrronium (e.g. as bromide), SVT-40776, CHF 4226 and quinuclidine derivatives as disclosed in US 2003/0055080.

The pharmaceutically active substance may, where applicable, be in the form of a salt, a solvate, or a solvate of a salt or in the form of a derivative, e.g. an ester derivative.

Furthermore, the pharmaceutically active substance may be capable of existing in stereoisomeric forms. It will be understood that the invention encompasses the use of all geometric and optical isomers (including atropisomers, enantiomers and diastereomers) of the pharmaceutically active substance and mixtures thereof including racemates. The use of tautomers and mixtures thereof also form an aspect of the present invention. Enantiomerically pure forms are particularly desired.

It will be appreciated that the dry powder formulations according to the invention may also contain other components such as taste masking agents, sweeteners, anti-static agents or absorption enhancers (e.g. sodium taurocholate). Where such component(s) is/are present, it/they will generally be present in a total amount not exceeding 10 percent by weight (% w) of the total weight of the composition.

The dry powder formulations according to the invention may be prepared by blending together a pharmaceutically active substance, an excipient and an additive being the reaction product of ascorbic acid with (i) a saturated or unsaturated, straight or branched C₁₂-C₁₈ fatty acid, (ii) a straight or branched C₈-C₁₈ alkyl or alkenyl mono ester of a dibasic acid, (iii) a straight or branched C₁₀-C₁₈ alkanoyl or alkenoyl N-substituted amino acid, or (iv) a straight or branched C₁₀-C₁₈ alkanoyl or alkenoyl ester of a hydroxy acid, in a single step process. However, advantageous results are obtained if a two-step process is followed whereby, in a first step, the excipient and the additive material are blended together to form a mixture and then, in a second step, the mixture from the first step is blended with the pharmaceutically active substance.

In an embodiment of the invention, the dry powder formulation is prepared by a process comprising,
(1) blending a coarse component of excipient with an additive being the reaction product of ascorbic acid with (i) a saturated or unsaturated, straight or branched C₁₂-C₁₈ fatty acid, (ii) a straight or branched C₈-C₁₈ alkyl or alkenyl mono ester of a dibasic acid, (iii) a straight or branched C₁₀-C₁₈ alkanoyl or alkenoyl N-substituted amino acid, or (iv) a straight or branched C₁₀-C₁₈ alkanoyl or alkenoyl ester of a hydroxy acid, to form a mixture, and
(2) blending the mixture obtained in step (1) with a pharmaceutically active substance and, optionally, a fine component of excipient.

Any kind of mixer can be used in the single step or the two-step process, for example, tumbling blenders such as the Turbula blender or the Bohle blender, planetary blenders, intensive mixers (Fielder, Colette, Bohle) or intensive mixers equipped with heating and/or vacuum generating means (Colette, Zanchetta). The mixing times and mixing speeds chosen will depend on a variety of factors including the type of blender used and the batch size. The mixing times will generally be in the range from 2 minutes to 120 minutes. In the two-step process, the mixing time for step 1 is preferably longer than the mixing time for step 2. The mixing is suitably carried out under relative humidity (RH) conditions ranging from dry to medium, that is, from 0 to 60% RH, and the temperature is suitably in the range from 0° C. to 60° C., preferably from 5° C. to 40° C.

Any suitable dry powder inhaler (DPI) may be used to deliver the dry powder formulations according to the invention. The DPI may be “passive” or breath-actuated, or “active” where the powder is dispersed by some mechanism other than the patient's inhalation, for instance, an internal supply of compressed air. At present, three types of passive dry powder inhalers are available: single-dose, multiple unit dose or multidose (reservoir) inhalers. In single-dose devices, individual doses are provided, usually in capsules, and have to be loaded into the inhaler before use, examples of which include Spinhaler® (Aventis), Rotahaler® (GlaxoSmithKline), Aeroliser™ (Novartis), Inhalator® (Boehringer) and Eclipse (Aventis) devices. Multiple unit dose inhalers contain a number of individually packaged doses, either as multiple gelatine capsules or in blisters, examples of which include Diskhaler® (GlaxoSmithKline), Diskus® (GlaxoSmithKline), Aerohaler® (Boehringer) and Handihaler® (Boehringer) devices. In multidose devices, drug is stored in a bulk powder reservoir from which individual doses are metered, examples of which include Turbuhaler® (AstraZeneca), Easyhaler® (Orion), Novolizer® (ASTA Medica), Clickhaler® (Innovata Biomed) and Pulvinal® (Chiesi) devices.

Thus, the present invention further provides a dry powder inhaler, in particular a multiple unit dose dry powder inhaler, containing a dry powder formulation of the invention as hereinbefore described.

The invention will now be further described by reference to the following illustrative examples.

### EXAMPLE 1

### Preparation of Dry Powder Formulations

The formulations I to IX containing the drug beclomethasone dipropionate (BDP) shown in Table 1 below were prepared according to the following procedure in which Steps 1 and 2 were performed under low relative humidity (RH) conditions, i.e., below 30% RH. Eight different additives were tested: ascorbyl palmitate obtained from Sigma-Aldrich Company, U.K. (6-O-palmitoyl-L-ascorbic acid, an additive according to the invention), palmitic acid obtained from Sigma-Aldrich Company, U.K. (comparison additive), glyceryl monostearate obtained from Faci, Italy (comparison additive), magnesium stearate obtained from Peter Greven, Germany (comparison additive), sucrose monostearate and sucrose monopalmitate obtained from Sisterna, Netherlands (comparison additives), ascorbyl octanoate (comparison additive) and ascorbyl dodecanoate (additive according to the invention)

The excipient used was lactose (inhalation grade sieved lactose monohydrate) as sold under the trade mark “Respitose SV003” by DMV International B.V., Veghel, Netherlands.

The batch size in each case was 200 grams. Batch compositions are given in Table 1.

### Step 1

Into a 1 litre mixing vessel fitted to a Diosna P1-6 intensive mixer was charged half of the lactose excipient followed by all of the additive and then the remaining half of the lactose excipient. The contents of the mixing vessel were mixed at 500 revolutions per minute (rpm) for one minute. The mixer was opened and the powder located on the upper walls of the mixing vessel was scraped down. Mixing was continued at 1500 rpm for two further periods of seven minutes each, with the upper walls of the mixing vessel being scraped down in between these mixing periods. The mixer was then opened and if the powder contained any lumps, it was sieved using a sieving machine fitted with a 1.0 mm sieve (by Retsch GmbH, Germany).

### Step 2

In the same mixer as used in Step 1, micronised BDP having a mass median diameter (MMD) below 5 μm was gently mixed together with the mixture obtained in Step 1 using a spoon. The resulting mixture was blended at 500 rpm for one minute. The mixer was opened and the powder on the upper walls of the mixing vessel was scraped down. Mixing was continued for two further periods of 7 minutes each at 1500 rpm with scraping down being carried out inbetween mixing periods. The powder formulation obtained was carefully emptied into a plastic container and stored under dry conditions (relative humidity less than about 30%).

When preparing the reference batch (Formulation I), the drug was added instead of the additive in Step 1 and Step 2 was omitted.

TABLE 1

| | | | Beclomethasone |
|---|---|---|---|
| | Lactose | | Dipropionate |
| Formulation | (% w)* | Additive (% w)* | (% w)* |
| I (reference) | 98 | — | 2.0 |
| II (comparison) | 97.5 | Palmitic acid (0.5) | 2.0 |
| III (comparison) | 97.5 | Glyceryl | 2.0 |
| | | monostearate (0.5) | |
| IV (invention) | 97.5 | Ascorbyl palmitate | 2.0 |
| | | (0.5) | |
| V (comparison) | 97.5 | Magnesium stearate | 2.0 |
| | | (0.5) | |
| VI (comparison) | 97.5 | Sucrose | 2.0 |
| | | monostearate (0.5) | |
| VII (comparison) | 97.5 | Sucrose | 2.0 |
| | | monopalmitate (0.5) | |
| VIII (comparison) | 97.5 | Ascorbyl octanoate | 2.0 |
| | | (0.5) | |
| IX (invention) | 97.5 | Ascorbyl | 2.0 |
| | | dodecanoate (0.5) | |

*all percentages by weight are based on the total weight of the formulation

### EXAMPLE 2

### Measurement of Fine Particle Fraction

Fine particle assessment was analysed using the Next Generation Impactor, NGI. This impactor is described in pharmacopoeias such as thee Eur. Pharmacopoeia (5.8 section 2.9.18, apparatus E) where there is a detailed description about how to set up, operate and calibrate the impactor for use at different flow rates.

A simple prototype inhaler was used consisting of an L-shaped cylindrical channel comprising a vertical component and a horizontal component. In addition there was a support with cylindrical holes for scrape filling the powder but this feature was not used. The device was fitted via a USP-inlet to the Next Generation Impactor. The powder, approximately 5 milligrams (mg), was transferred to the vertical channel into the bend of the device, i.e. the bend of the L-shaped channel. An airflow pulse (see below) then activated the airflow through the device, entraining the powder located in the bend, and the air/particle mixture thereafter moved through the horizontal component of the channel and into the Next Generation Impactor.

Each dose of approximately 5 mg was drawn with an airflow pulse of duration 3.1 seconds at a flow rate of 77 l/min through the device. The impactor steps were then analysed for drug content and the fine particle dose was obtained.

The fine particle fraction was calculated as the fine particle dose divided by the total amount of drug per dose delivered to the NGI. The results are shown in Table 2. It is evident that the addition of ascorbyl palmitate (see Formulation IV according to the invention) gave rise to a dramatic increase in the fine particle fraction as compared to the reference formulation (Formulation I) without additive, whilst several of the additives (see comparison Formulations II, III, VI, VII and VIII) made no improvement at all to the fine particle fraction. The addition of magnesium stearate (which is well known from the literature; Formulation V) showed only a modest improvement in the fine particle fraction that was less than half that obtained using ascorbyl palmitate according to the invention.

TABLE 2

| Formulation | Additive used | Fine particle fraction (%) |
|---|---|---|
| I (reference) | — | 3.9 |
| II (comparison) | Palmitic acid | 2.9 |
| III (comparison) | Glyceryl monostearate | 2.2 |
| IV (invention) | Ascorbyl palmitate | 24.8 |
| V (comparison) | Magnesium stearate | 11.9 |
| VI (comparison) | Sucrose monostearate | 2.8 |
| VII (comparison) | Sucrose monopalmitate | 2.7 |
| VIII (comparison) | Ascorbyl octanoate | 2.5 |
| IX (invention) | Ascorbyl dodecanoate | 6.6 |

### EXAMPLE 3

Dry powder formulations according to the invention containing 5% w BDP and different amounts of ascorbyl palmitate additive, as shown in Table 3, were prepared according to the procedure described in Example 1 above. A new reference batch (Formulation X) was prepared in the same way as Formulation I above.

TABLE 3

| | | | Beclomethasone |
|---|---|---|---|
| | | Ascorbyl Palmitate | Diproprionate |
| Formulation | Lactose (% w)* | (% w)* | (% w)* |
| X | 95 | — | 5.0 |
| XI | 94.5 | 0.5 | 5.0 |
| XII | 94.0 | 1.0 | 5.0 |
| XIII | 93.0 | 2.0 | 5.0 |
| XIV | 90.0 | 5.0 | 5.0 |
| XV | 85.0 | 10.0 | 5.0 |
| XVI | 80.0 | 15.0 | 5.0 |
| XVII | 75.0 | 20.0 | 5.0 |

*all percentages by weight are based on the total weight of the formulation

The fine particle fractions of the formulations in Table 3 were measured according to the procedure described in Example 2 and the following values were obtained:

TABLE 4

| | Ascorbyl palmitate | Fine particle fraction |
|---|---|---|
| Formulation | (% w)* | (%) |
| X | — | 3.6 |
| XI | 0.5 | 15.1 |
| XII | 1.0 | 23.9 |
| XIII | 2.0 | 14.7 |
| XIV | 5.0 | 20.8 |
| XV | 10.0 | 35.8 |
| XVI | 15.0 | 33.9 |
| XVII | 20.0 | 36.1 |

*all percentages by weight are based on the total weight of the formulation

### EXAMPLE 4

Dry powder formulations according to the invention containing 5% w of either salbutamol sulphate (SBS) or budesonide (BUD) and 10% w ascorbyl palmitate additive, as shown in Table 5, were prepared according to the procedure described in Example 1 above.

TABLE 5

| | | Ascorbyl Palmitate | Drug Substance |
|---|---|---|---|
| Formulation | Lactose (% w)* | (% w)* | (% w)* |
| XVIII | 95.0 | — | SBS (5.0) |
| XIX | 85.0 | 10.0 | SBS (5.0) |
| XX | 95.0 | — | BUD (5.0) |
| XXI | 85.0 | 10.0 | BUD (5.0) |

*all percentages by weight are based on the total weight of the formulation

The fine particle fractions of the formulations in Table 5 were measured according to the procedure described in Example 2. The results obtained are shown in Table 6 below and are compared alongside the results obtained for similar formulations containing BDP (Formulations X and XV from Example 3).

TABLE 6

| | Drug | Ascorbyl Palmitate | Fine particle |
|---|---|---|---|
| Formulation | Substance | (% w)* | fraction (%) |
| X | BDP | — | 3.6 |
| XV | BDP | 10.0 | 35.8 |
| XVIII | SBS | — | 25.1 |
| XIX | SBS | 10.0 | 43.5 |
| XX | BUD | — | 20.2 |
| XXI | BUD | 10.0 | 45.9 |

*all percentages by weight are based on the total weight of the formulation

The lipophilicity/hydrophilicity of the drugs BDP, SBS and BUD are quite different to one another. Budesonide is a rather lipophilic drug with a water solubility of 16 μg/ml at 25° C. and BDP is a very lipophilic drug with a water solubility of 0.13 μg/ml at 25° C. whereas SBS is a hydrophilic, highly water-soluble drug.

The results in Table 6 clearly show that the addition of ascorbyl palmitate leads to a significant improvement in the fine particle fraction of the dry powder formulation irrespective of the type of drug present.

### EXAMPLE 5

Dry powder formulations were prepared by the procedure described in Example 1 above which additionally contained a fine excipient component (micronised lactose monohydrate particles having an MMD less than 5 μm). The micronised lactose monohydrate was added at the same time as the micronised drug substance in the manufacture of the formulations. The compositions of the formulations prepared are shown in Table 7.

TABLE 7

| | | Fine | | |
|---|---|---|---|---|
| | Coarse | (micronised) | | |
| | lactose | lactose | | Ascorbyl |
| | component | component | Drug substance | Palmitate |
| Formulation | (% w) | (% w) | BDP (% w) | (% w) |
| XXII | 90.0 | 8.0 | 2.0 | — |
| XXIII | 89.5 | 8.0 | 2.0 | 0.5 |
| XXIV | 80.0 | 8.0 | 2.0 | 10.0 |

The fine particle fractions for the three formulations, when tested as described in Example 2 above, are given in Table 8.

TABLE 8

| | | Ascorbyl | Fine particle fraction |
|---|---|---|---|
| Formulation | Drug substance | Palmitate (% w) | (%) |
| XXII | BDP | — | 21.4 |
| XXIII | BDP | 0.5 | 38.9 |
| XXIV | BDP | 10.0 | 47.3 |

### EXAMPLE 6

Quick dissolution of the active drug substance is a prerequisite for rapid onset of action for inhalation drugs. In this example three different formulations were tested for dissolution kinetics using the beta-agonist salbutamol sulfate. All formulations were manufactured according to Example 1 above. The first formulation is a reference batch without additive whilst the second and third formulations contained 10% w of ascorbyl palmitate and 10% w of magnesium stearate, respectively. The total compositions are given in Table 9.

TABLE 9

| | Lactose | | Additive |
|---|---|---|---|
| Formulation | (% w) | SBS (% w) | (% w) |
| XXV | 90.0 | 10.0 | — |
| XIX | 85.0 | 5.0 | Ascorbyl |
| | | | palmitate (10.0) |
| XXVI | 85.0 | 5.0 | Magnesium |
| | | | stearate (10.0) |

*AP = Ascorbyl palmitate, MgSt = Magnesium stearate

For determination of the dissolution rate, a fiber optic dissolution system measuring the change in UV-absorption in the dissolution media was used (μDiss Profiler, Pion Inc. MA). This system consists of an optical measurement unit, comprising in situ sample probes, a UV/DA-detection system (one detector per probe) and a UV-lamp, plus a sample holder assembly. The sample holder assembly consists of holders for 30 ml vials with a heat block and a magnetic stirring device. It is possible to adjust the size of the probe aperture (i.e. the optical path length in the dissolution media), to facilitate measurements over a broader absorption interval. In this experiment it was set to 5 mm.

A standard solution of SBS was prepared. The substance was dissolved in a solvent, where the solubility of the substance is significantly higher compared to the dissolution media used. These solvents do not absorb UV-radiation in the wavelength interval used for the measurements. The system was calibrated by adding known volumes of standard solution to the same type of media used for the dissolution experiment (phosphate buffer pH 7 with 1 mM sodium dodecylsulfate). Typically, the volume ratio between added standard solution and dissolution media during calibration did not exceed 5%.

Before measurement, all probes were submersed in dissolution media and the background absorption was measured. The media was removed and the probes were placed in sample vials containing weighed amounts of sample powder. The amount of formulation was chosen so as to give the same total amount of SBS. 16 mg per vial were used for formulations XIX and XXVI and 8 mg per vial for formulation XXV. Directly after the UV-measurement was started, 20 ml of dissolution media was added to each sample vial. A magnetic stirrer was continuously stirring at 300±1 rpm in the bottom of the sample vial. The dissolution was traced until there was no change in the bulk concentration (i.e. when all particles had been dissolved or when the solubility limit had been reached).

All analyses were performed in duplicate. The temperature was set to 37° C. during the experiment. The absorbance range between 270 and 290 nm was used to calculate SBS concentrations. As ascorbyl palmitate has significant absorption overlapping with the absorbance of SBS, multivariate analysis of the UV-absorption in the entire wavelength range 220-390 nm was performed for Formulation XIX in order to resolve the dissolution of SBS.

Results from the dissolution tests, expressed as percent of SBS dissolved after 15 seconds and after 2 and 4 minutes are given in Table 10. It will be observed that SBS dissolves very rapidly in Formulation XXV and also quite rapidly in the formulation containing ascorbyl palmitate, Formulation XIX. Formulation XXVI containing magnesium stearate, on the other hand, gave a relatively slow dissolution of SBS.

| | | | | % of SBS | % of SBS |
|---|---|---|---|---|---|
| | | | % of SBS | dissolved | dissolved |
| Formu- | | Additive | dissolved | after 2 | after 4 |
| lation | Additive* | (% w) | after 15 sec* | minutes* | minutes* |
| XXV | — | — | 100 | 100 | 100 |
| XIX | Ascorbyl | 10.0 | 68 | 100 | 100 |
| | palmitate | | | | |
| XXVI | Magnesium | 10.0 | 35 | 63 | 80 |
| | stearate | | | | |

*Average of two tests

1. A dry powder formulation for use in inhalation therapy comprising a pharmaceutically active substance, an excipient and an additive being the reaction product of ascorbic acid with (i) a saturated or unsaturated, straight or branched C₁₂-C₁₈ fatty acid, (ii) a straight or branched C₈-C₁₈ alkyl or alkenyl mono ester of a dibasic acid, (iii) a straight or branched C₁₀-C₁₈ alkanoyl or alkenoyl N-substituted amino acid, or (iv) a straight or branched C₁₀-C₁₈ alkanoyl or alkenoyl ester of a hydroxy acid.

2. A dry powder formulation for use in inhalation therapy comprising a pharmaceutically active substance, an excipient and an additive being the reaction product of ascorbic acid with (i) a saturated or unsaturated, straight or branched C₁₂-C₁₈ fatty acid, (ii) a straight or branched C₈-C₁₈ alkyl or alkenyl mono ester of a dibasic acid, (iii) a straight or branched C₁₀-C₁₈ alkanoyl or alkenoyl N-substituted amino acid, or (iv) a straight or branched C₁₀-C₁₈ alkanoyl or alkenoyl ester of a hydroxy acid, provided that the excipient is not a cyclodextrin or any derivative thereof.

3. A dry powder formulation according to claim 1 or claim 2, wherein the additive is the reaction product of ascorbic acid with a saturated, straight chain C₁₂-C₁₈ fatty acid.

4. A dry powder formulation according to claim 3, wherein the additive is ascorbyl dodecanoate, ascorbyl myristate, ascorbyl palmitate or ascorbyl stearate.

5. A dry powder formulation according to claim 3, wherein the additive is ascorbyl palmitate.

6. A dry powder formulation according to claim 1 or claim 2, wherein the additive is present in an amount from 0.5 to 10% w based on the total weight of the formulation.

7. A dry powder formulation according to claim 1 or claim 2, wherein the excipient is glucose, galactose, D-mannose, arabinose, sorbose, lactose, maltose, sucrose, trehalose, mannitol, maltitol, xylitol, sorbitol, myo-inositol or erythritol, or a solvate of any one thereof.

8. A dry powder formulation according to claim 7, wherein the excipient is lactose monohydrate.

9. A dry powder formulation according to claim 8, wherein the excipient is erythritol.

10. A dry powder formulation according to claim 1 or claim 2, wherein the pharmaceutically active substance is a glucocorticosteroid, a long-acting β₂ agonist or an anticholinergic compound.

11. Use of an additive being the reaction product of ascorbic acid with (i) a saturated or unsaturated, straight or branched C₁₂-C₁₈ fatty acid, (ii) a straight or branched C₈-C₁₈ alkyl or alkenyl mono ester of a dibasic acid, (iii) a straight or branched C₁₀-C₁₈ alkanoyl or alkenoyl N-substituted amino acid, or (iv) a straight or branched C₁₀-C₁₈ alkanoyl or alkenoyl ester of a hydroxy acid, in a dry powder formulation for use in inhalation therapy in order to increase fine particle dose.

12. A dry powder inhaler containing a dry powder formulation as claimed in any one of claims 1 to 2.

13. A dry powder inhaler according to claim 12, wherein the inhaler is a multiple unit dose device.

14. A carrier material suitable for use in a dry powder pharmaceutical formulation comprising an excipient mixed with an additive being the reaction product of ascorbic acid with (i) a saturated or unsaturated, straight or branched C₁₂-C₁₈ fatty acid, (ii) a straight or branched C₈-C₁₈ alkyl or alkenyl mono ester of a dibasic acid, (iii) a straight or branched C₁₀-C₁₈ alkanoyl or alkenoyl N-substituted amino acid, or (iv) a straight or branched C₁₀-C₁₈ alkanoyl or alkenoyl ester of a hydroxy acid.

15. A process for preparing a dry powder formulation as defined in claim 1 which comprises, in a first step, blending excipient and additive to form a mixture and then, in a second step, blending the mixture obtained from the first step with a pharmaceutically active substance.
